(19) United States
(12) Patent Application Publication
(10) Pub. No.: US 2024/0428912 A1
(43) Pub. Date: Dec. 26, 2024

(54) HEALTH RECOMMENDER SYSTEM AND METHOD

(71) Applicant: Adhera Health, Inc., Santa Cruz, CA (US)

(72) Inventors: Luis FERNANDEZ-LUQUE, Sevilla (ES); Ricardo C. BERRIOS, Santa Cruz, CA (US); Santiago HORS-FRAILE, Sevilla (ES)

(73) Assignee: Adhera Health, Inc., Santa Cruz, CA (US)

(21) Appl. No.: 18/829,869

(22) Filed: Sep. 10, 2024

Related U.S. Application Data
(63) Continuation-in-part of application No. PCT/US2023/015030, filed on Mar. 11, 2023.
(60) Provisional application No. 63/269,246, filed on Mar. 11, 2022.

(51) Int. Cl. G16H 20/00; G16H 10/20

(52) U.S. Cl. CPC G16H 20/00; G16H 10/20

(57) ABSTRACT

A method for supporting self-management of people with at least one chronic condition, and the assessment of their psychical and mental conditions is provided. The method includes a health recommender and is carried out by at least one processor. The method includes receiving by the at least one processor first and second health parameters of a user, and additional data describing the interactions of the user with previous data generated by the health recommender. The method also includes receiving by the at least one processor content data, and processing by the at least one processor the first and second health parameters to generate a first user model, and generating by the at least one processor at least one health recommendation to the user comprising content data. The method then includes selecting at least one recommendation for the user and providing the selected at least one recommendation to the user.

### RELATED APPLICATIONS

This application is a bypass continuation-in-part of International Application No. PCT/US2023/015030 filed Mar. 11, 2023, which claims priority to U.S. Application No. 63/269,246 filed Mar. 11, 2022, each of which is hereby incorporated herein by reference in its entirety.

### FIELD OF THE INVENTION

The present disclosure relates generally to methods and systems for the assessment and management of personal health and more particularly to a multi-dimensional health recommendation system for precise assessment and management of lifestyle-related symptoms of chronic conditions.

### BACKGROUND OF THE DISCLOSURE

Interventions to improve self-management including promoting mental health and healthier lifestyles of people with chronic conditions and their family caregivers have been proven to be effective in reducing the impact of many symptoms in people living with chronic conditions. For example, interventions aiming at improving mental wellbeing (i.e., mindfulness, Cognitive Behavioral Therapy, etc.) and promoting physical activity have been found to be effective for reducing insomnia symptoms and chronic-condition-related fatigue.

Current recommender systems for patients do not keep healthcare professionals in the loop of the system as another potential channel for the distribution of recommendations, and this poses a major challenge due to potential mistrust of the system and the lack of coherence between clinicians and the health recommender system.

The success of self-management interventions aiming at both mental and behavioral aspects are more effective when personalized to the unique health context of each patient, including emotional status, lifestyle, quality of sleep, diagnosis, and medications. However, clinicians involved in supporting patients with chronic conditions affected by different symptoms lack information on the multidimensional lifestyle and behavioral aspects in between the clinical visits. This puts a major barrier to the persuasiveness of the healthcare professionals' recommendations due to the lack of deep understanding of the patients' needs, and also the physical inability to provide the right recommendations at the right time and place.

The prior art in health recommender systems fails to address the comprehensive context of the patients for its user modeling (e.g., engagement with the digital health solution, objective and subjective health data) and consequently has limited personalization potential.

Health recommendations that are not contextualized are less trustworthy and persuasive. Traditional recommender systems do not provide explanations taking into account the subjective and objective health of the patients.

Current developments in recommender systems allow for context-aware recommendations including in the health domain, but those are not considering the multi-dimensional and dynamic needs of the patients affected by multiple symptoms. Each patient is affected uniquely by a constellation of health symptoms and has unique behavioral needs. Further, health recommender systems traditionally only provide a single modality of recommendations (e.g., motivational messages) and do not address the multi-modality of digital interventions that might combine multiple types of therapeutic contents (e.g., CBT exercises, educational content, behavioral messages).

The prior art fails to teach a method that enables behavioral and mental health changes to support the self-management of symptoms of chronic conditions by automatic monitoring and modeling patient's context and that provides recommendations of content adapted to the context of the patient.

The prior art in health recommender systems fails to consider the required adaptations to the different phases within the patient journey, using the same approach for the entire patient journey rather than contextualizing for specific health problems across the patient journey or health programs.

The prior art in health recommender systems fails to integrate elements into the healthcare delivery, such as structured programs that combine education, motivational aspects, and interaction with healthcare professionals.

### SUMMARY OF THE INVENTION

Disclosed herein is a method and system for supporting self-management of people with chronic conditions by adapting to the unique and evolving physical and mental health by automatic monitoring and modeling of a patient's context and for providing recommendations of content adapted to the context of the patient. These recommendations of content can be provided directly by the system or via a healthcare professional who is engaging with the patient. The recommendations are further contextualized with health data and trends visualizations aiming to increase the explainability of the recommendations.

The embodiments disclosed herein are only examples of the many possible advantageous uses and implementations of the innovative teachings presented herein. In general, statements made in the specification of the present disclosure do not necessarily limit any of the various claimed inventions. Moreover, some statements may apply to some inventive features but not to others. In general, unless otherwise indicated, singular elements may be in plural and vice versa with no loss of generality. In the drawings, numerals refer to like parts through several views.

The term “mobile device” and similar, as used herein is intended to include but not be limited to any of the following: mobile telephone, smartphone, PDA, smartwatch, iPad, game console, tablet, laptop, or another computer terminal, and embedded remote unit.

The term “sensor” and similar, as used herein is intended to include but not be limited to any of the following: wearable sensors, audio sensors (e.g., microphone, an acoustic transducer, etc.), video sensor (e.g., video camera, IR camera, gesture sensor, etc.), weight sensor (e.g., scale, BMI sensor, physiological sensors, and implantable sensors).

The term “health data” and similar, as used herein is intended to include but not be limited to any of the following: Electronic health records, self-reported health data, therapeutic data, clinical data, psychological reports, psychiatric reports, behavioral and mental health data, health data provided by personal devices (e.g., wearable sensors), sensors that are used as “digital biomarker” of health-related measurements (e.g., depression assessment from wearables), etc. Health data provided by personal devices may be online data (i.e., reported in real-time) or offline (i.e., recorded data). The health data also includes demographics and other social-related factors such as education and cultural preferences. For example, the health data may include biopsychosocial models of data which consider the interconnection between biological, psychological, and socio-environmental factors (e.g., education level, income level, neighborhood and mother tongue) and specifically how these aspects play a role in health. The health data can be related to both the patients and/or their family caregivers. The health data can also include family interpersonal dynamics (e.g., communication issues between family caregiver and the patient).

The term “engagement data” and similar, as used herein is intended to include but not be limited to any of the following: the interactions of the users with the different elements of the digital solutions including ratings of content, views of content, and any other interactions with elements available in the user interface.

The term “patient” refers to the person affected by a chronic condition, who might be the patient themselves or his family caregiver (e.g. sibling, spouse or friend) who might also have his health affected due to the burden of caring.

The term “user model” and similar, as used herein is intended to include but not be limited to any of the following: the combination of data that belongs to an individual and it is used for modeling proposes, that includes health data and engagement data.

The term “health meta-feature”, as used herein is intended to include but not be limited to any of the following: meta-data which describe characteristics of therapeutic and sensing content with regards to their design towards addressing aspects related to particular behavioral and mental health factors (e.g., content designed for users with low mood) and also physical health (e.g., content designed for users with a particular type of surgery). These meta-features also address related social health factors when relevant (e.g., relationships, family support, cultural factors, economic factors, education). These meta-features combined represent a semantic-space and a taxonomy of the contents of the Health Recommender System.

The term “health-gram”, as used herein is intended to include but not be limited to any of the following: the longitudinal representation of all health data related to the users, including, among others, the subjective data captured subjectively by asking the user (e.g., the question “how do you feel today?”) and objective data (e.g., number of steps) that can come from both sensors and other relevant data sources (e.g., prescribed medications).

The term “recommendation” and similar, as used herein is intended to include but not be limited to any of the following: recommendations of therapeutic content which are tailored to the individual unique health needs and aiming at supporting the self-management of the patients, including both mental and physical health aspects such as lifestyle recommendations, behavioral and emotional support. The recommendations may also include sensing content (e.g., quizzes and psychometrics) that are provided to users at the right time and place. Recommendations may also include both the selection for the right content and also its adaptation (e.g., modification of the content to further personalize using specific patient's data).

The term “objective measurement” or “objective data” and similar, as used herein is intended to include but not be limited to any of the following: measurements that are not influenced by the opinion or perspective of people, such as age, lab value results, and sensor data. Objective data may also include information extracted from EMR (electronic medical records) as provided by medical systems.

The term “subjective measurement” and similar, as used herein is intended to include but not be limited to any of the following: measurements that are inherently depending on the perspective of the person making the measurement, that includes psychometrics such as perceived tiredness or mood.

The term “therapeutic content” and similar, as used herein is intended to include but not be limited to any of the following: refers to digital content that has been designed to provide a therapeutic value (aka supporting patient self-management), that therapeutic content might include motivational messages, mental wellbeing exercises, educational content among others. The therapeutic content does contain metadata with relevant health meta-features. The content model includes both the content itself (e.g., educational video) and the metadata describing the health and behavioral meta-features which define the characteristics for which the content is used (e.g., whether the content is designed for low mood patients).

According to an aspect of the present disclosure, a system and a method orchestrates a health recommender system aligned with a precision digital therapeutics program. Such an approach may allow creating personalized recommendations that may increase patient engagement and may also reinforce therapeutic intervention programs.

According to an embodiment of one or more paragraph(s) of this disclosure, there is provided a health recommender system and method that may address the comprehensive context of the patients for its user modeling (e.g., engagement with the digital health solution, objective and subjective health data).

According to an embodiment of one or more paragraph(s) of this disclosure, the health recommender system and method may enable behavioral and mental health changes to support the self-management of symptoms of chronic conditions by automatic monitoring and modeling patient's context which may provide recommendations of content adapted to the context of the patient. According to some embodiments, the health recommender may consider the required adaptations to the different phases within the patient journey, by using different approaches for contextualizing for specific health problems across the patient journey or health programs.

According to an embodiment of one or more paragraph(s) of this disclosure, the health recommender system and method may integrate elements into the healthcare delivery, such as structured programs that may combine education, motivational aspects, and interaction with healthcare professionals.

According to an aspect of the present disclosure, there is provided a computer implemented method for supporting self-management of people with at least one chronic condition, and supporting the assessment of their psychical and mental conditions, wherein said method further includes a health recommender, the method carried out by at least one processor, said method comprising:
a) receiving, by the at least one processor, a plurality of first health parameters of a user, wherein said first health parameters denote the user's objective data (i.e., wearable and sensor data) and wherein at least one of the first health parameters represents data measured by an external device (e.g., wearable device, sensor, etc.);
b) receiving, by the at least one processor, a plurality of second health parameters of a user, wherein said second health parameters include at least one subjective measurement; wherein said at least one subjective measurement denotes a perception of the user's subjective health;
c) receiving, by the at least one processor, additional data describing the interactions of the user with previous data generated by the health recommender;
d) receiving, by the at least one processor, content data, said content data comprising information relevant to the self-management of chronic conditions and characteristics of the recommendable content;
e) processing, by the at least one processor, the first and second health parameters to generate a first user model, wherein the first user model describes the user characteristics; and wherein the at least one processor has further access to additional user models previously generated for people with at least one chronic condition;
f) generating, by the at least one processor, at least one health recommendation to the user comprising content data, said recommendation based on the first user model, the content data and, the other user models;
g) selecting at least one recommendation for the user; and
h) providing the selected at least one recommendation to the user.

According to an embodiment of one or more paragraph(s) of this disclosure, the at least one processor may be configured to communicate with at least one external device and wherein the external device may be configured to exchange data (e.g., using wired or wireless communication channels) with the at least one processor.

According to an embodiment of one or more paragraph(s) of this disclosure, the second health parameters may further include at least one subjective measurement, and wherein the subjective measurement may include at least one of: psychometric parameter, questionnaire results, quiz, and the like.

According to an embodiment of one or more paragraph(s) of this disclosure, the processor may have further access to previous recommendations provided by the health recommender (e.g., stored in a database which may be a local database or a database in the cloud) and wherein generating the health recommendation may further include the interactions of the patient with at least one previous recommendation of the health recommender. According to some embodiments, the processor may be also able to retrieve historical data of any type including objective and subjective data, users' models, unique individual health data of the user, and health context of other users with similar characteristics, etc.

According to an embodiment of one or more paragraph(s) of this disclosure the method may also include a process of reducing the health data dimension by the health recommender, wherein said reducing includes consideration of the unique individual health context of the user and health context of other users with similar characteristics, and wherein said reducing is performed before said generating.

According to an embodiment of one or more paragraph(s) of this disclosure, the method may further include the use of artificial intelligence (e.g., machine learning, supervised learning, reinforcement learning, unsupervised learning, deep learning, etc.). In some embodiments, the health recommender system may include means to allow machine learning training. This may include training the computer model to different patient/user models, different objective (e.g., electronic medical records) and subjective data, symptoms predicted trends, the lifestyle of the patient, and the like. According to some embodiments, at least one function performed by the at least one processor (e.g., generating a recommendation) may further include the use of machine learning and artificial intelligence.

According to an embodiment of one or more paragraph(s) of this disclosure, providing the recommendations may further include tailoring the selected recommendation to include the personal characteristics of the user. According to some embodiments, tailoring the selected at least one recommendation may further include adapting the selected at least one recommendation to the user, by combining artificial intelligence models about symptoms predicted trends and their contributing factors.

According to an embodiment of one or more paragraph(s) of this disclosure, the first and second health parameters may further include weighing factors, and wherein processing those parameters may take into consideration the weighing factors to generate the first user model.

According to an embodiment of one or more paragraph(s) of this disclosure, the method for supporting self-management of people with at least one chronic condition, and to support the assessment of their psychical and mental conditions may further include modeling of therapeutic content addressing specific behavioral and mental health strategies to reduce the symptom burden of people living with chronic conditions.

According to an embodiment of one or more paragraph(s) of this disclosure, the method may further incorporate ratings of recommendations by clinicians to further improve the efficacy of the recommendations.

According to an embodiment of one or more paragraph(s) of this disclosure, the therapeutic approach of the method for the management and assessment of the health of a user (e.g., a patient) may be based on a:
Program-based health recommender configuration: a configuration of the health recommender system for a determined health program (e.g., program to support cancer-related fatigue) that includes, among others, the determination of which relevant contents are available for recommendations within the time-bounded program.
Enhanced Sensing: a combination of subjective sensing (aka psychometrics) with objective data (i.e., wearable data) that may allow a deeper understanding of the overall physical and mental health status of the users/patients. The semantic integration of those two types of data on the user models may be crucial when used by the health Recommender System and also by a health decision support system (e.g., Adhera Health Collaboration system).

According to another aspect of the present disclosure, there is provided a health recommender system to support the self-management of people with at least one chronic condition, including their family caregiver when relevant, and to support the assessment of their physical and mental conditions, the system comprising:
at least one processor comprising at least one communication channel configured to communicate with external devices to receive and store a plurality of first health parameters of a user, wherein said first health parameters denote user's objective data and wherein at least one of the first health parameters represents data measured by an external device;
and wherein said at least one processor is further configured receive a plurality of second health parameters of a user, wherein said second health parameters include at least one subjective measurement; wherein said at least one subjective measurement denotes a perception of the user's subjective health;
and wherein said at least one processor is further configured to receive additional data describing the interactions of the user with previous data generated by the health recommender; and wherein said at least one processor is further configured to receive content data comprising information relevant to the self-management of chronic conditions and characteristics of the recommendable content; and
access to a storage device to store and retrieve the plurality of first health parameters, the plurality of second health parameters of a patient, the content data, and the additional data;
wherein the at least one processor is configured to perform functions related to the first health parameters, the second health parameters, the content data and the additional data, said functions comprising:
   a) receiving, by the at least one processor, a plurality of first health parameters of a user, wherein said first health parameters denote the user's objective data and wherein at least one of the first health parameters represents data measured by an external device;
   b) receiving, by the at least one processor, a plurality of second health parameters of a user, wherein said second health parameters include at least one subjective measurement; wherein said at least one subjective measurement denotes a perception of the user's subjective health;
   c) receiving, by the at least one processor, additional data describing the interactions of the user with previous data generated by the health recommender;
   d) receiving, by the at least one processor, content data, said content data comprising information relevant to the self-management of chronic conditions and characteristics of the recommendable content;
   e) processing, by the at least one processor, the first and second health parameters to generate a first user model, wherein the first user model describes the user characteristics; and wherein the at least one processor has further access to additional user models previously generated for people with at least one chronic condition;
   f) generating, by the at least one processor, at least one health recommendation to the user comprising content data, said recommendation based on the first user model, the content data and, the other user models;
   g) selecting at least one recommendation for the user; and
   h) providing the selected at least one recommendation to the user.

According to an embodiment of one or more paragraph(s) of this disclosure, the system may include at least one processor which may communicate with at least one external device and wherein said external device may exchange data with the at least one processor.

According to an embodiment of one or more paragraph(s) of this disclosure, the second health parameters may include at least one subjective measurement, and wherein said subjective measurement may include at least one of: psychometric parameters, questionnaire results, and the like.

According to an embodiment of one or more paragraph(s) of this disclosure, at least one processor may have further access to previous recommendations provided by the health recommender and wherein generating a recommendation may further include the interaction of the patient with at least one previous recommendation of the health recommender.

According to an embodiment of one or more paragraph(s) of this disclosure, the health recommender may further reduce the health data dimension, wherein said reduction may consider the unique individual health context of the user and health context of other users with similar characteristics, and wherein said reduction may be performed before generating the recommendation.

According to an embodiment of one or more paragraph(s) of this disclosure, at least one of the functions (e.g., generating a recommendation) performed by the at least one processor may further include the use of machine learning and artificial intelligence.

According to an embodiment of one or more paragraph(s) of this disclosure, providing a recommendation may further include tailoring the selected recommendation to include personal characteristics of the user. For example, according to some embodiments, said tailoring may further include adapting the selected at least one recommendation to the user, by combining artificial intelligence models about symptoms predicted trends and their contributing factors.

According to an embodiment of one or more paragraph(s) of this disclosure, said first and second health parameters may further include weighing factors and wherein said processing may consider said weighing factors to generate the first user model.

According to another aspect of the present disclosure, there is provided a non-transitory computer readable storage medium in a system comprising at least one processor with at least one communication channel configured to communicate with external devices, a memory and, access to a storage device to store and retrieve data, wherein said computer readable storage medium stores at least one readable program, wherein said at least one program supports the self-management of people with at least one chronic condition and supports the assessment of their psychical and mental conditions, and when said at least one program is executed by the at least one processor causes the at least one processor to:
a) receive, by the at least one processor, a plurality of first health parameters of a user, wherein said first health parameters denote user's objective data and wherein at least one of the first health parameters represents data measured by an external device;
b) receive, by the at least one processor, a plurality of second health parameters of a user, wherein said second health parameters include at least one subjective measurement; wherein said at least one subjective measurement denotes a perception of the user's subjective health;
c) receive, by the at least one processor, additional data describing the interactions of the user with previous data generated by the health recommender;
d) receive, by the at least one processor, content data, said content data comprising information relevant to the self-management of chronic conditions and characteristics of the recommendable content;
e) process, by the at least one processor, the first and second health parameters to generate a first user model, wherein the first user model describes the user characteristics; and wherein the at least one processor has further access to additional user models previously generated for people with at least one chronic condition;
f) generate, by the at least one processor, at least one health recommendation to the user comprising content data, said recommendation based on the first user model, the content data and, the other user models;
g) select at least one recommendation for the user; and
h) provide the selected at least one recommendation to the user.

According to an embodiment of one or more paragraph(s) of this disclosure, a patient with multiple sclerosis may be prescribed to join an intervention to address both mental and physical fatigue symptoms. Accordingly, the Health Recommender system may use a configuration of the Health Recommender System (HRS) for Chronic-condition Related Fatigue Program (CCRF) which:
The therapeutic programs for CCRF include addressing the contribution of mental health symptoms with a) Mindfulness and CBT, b) therapeutic educational content, c) behavioral motivational messages towards improving adherence to protective behavior. The program is designed to be guided for 12 weeks with more extensive support from healthcare professionals, and self-guided as open-ended with very limited interaction with healthcare professionals.
The HRS provides recommendations for those two phases and the recommendations also rely on the prediction of individual factors based on the application of machine learning or artificial intelligence for trend prediction of relevant health parameters.

According to an embodiment of one or more paragraph(s) of this disclosure, the method for the management and assessment of the health of a patient addresses the main challenges of providing contextually meaning therapeutic content aiming at supporting self-management of people with chronic conditions. A key challenge in supporting people with chronic conditions may be the need to provide tailored feedback that may address their unique combination of symptoms and contributing factors.

As may be apparent to the skilled in the art, the method relies on engagement data (aka interaction between user and therapeutic content) which is combined with health status user modeling (inferred from psychometrics and sensor data). This may create a multi-dimension user and therapeutic content matrix which encompasses all the contextual dimensions.

According to an embodiment of one or more paragraph(s) of this disclosure, using data analytics techniques allows performing a dimensionality reduction thus providing a pre-contextualized user-item matrix which may be used as the starting point of a hybrid recommender system.

According to an embodiment of one or more paragraph(s) of this disclosure, as part of the hybrid recommender system, a heuristic is further applied for the transition between knowledge-based recommendations (i.e., to avoid cold start problems) and fully collaborative-based recommendations.

According to an embodiment of one or more paragraph(s) of this disclosure, the recommendations may be further sent to the user depending on the temporal context and user's device (e.g., cellphone, wearable device, smart speaker, tablets, etc.). That may include just-in-time recommendations that are provided after detecting a health event (i.e., filling a psychometric, performing physical activity, waking up, etc.).

According to an embodiment of one or more paragraph(s) of this disclosure, clinicians may prompt recommendations for patients which they can generate an encounter (virtual or physical) with said patients. If one or more recommendations are provided to the patient, the clinician may mark those recommendations as “delivered”. According to some embodiments, this “delivered” marking may also be considered as an implicit rating, or explicit if the patient further agreed to try out.

According to an embodiment of one or more paragraph(s) of this disclosure, the knowledge-based prefiltering of recommendations may be based on one or more health characteristics (e.g., as may be defined in the Adhera Health Therapeutic programs). Said knowledge-based prefiltering may provide recommendations that are already aligned with the overall health program. For example, and according to some embodiments, the Adhera Health Program for Stress Management for patients undergoing Surgery may include some rules that determine that some educational content is only recommended for a specific type of surgery.

According to an embodiment of one or more paragraph(s) of this disclosure, these programs may cover a large variety of health-related aspects. For example, the Adhera for Fatigue and Mental Health program includes “configuration” aspects in respect to the following dimensions related to fatigue contributing factors:
Definition of eligible users: chronic conditions, levels of perceived fatigue.
Active and maintenance phase of the program: the active part of the program is the number of weeks during which the user has access to a more intensive program with a more intensive number of recommendations and enhanced support from clinicians. This may be followed by an open-ended period with fewer recommendations and reduced clinical support.
Behavioral and lifestyle meta-features: refers to behavioral and lifestyle factors that are addressed to help patients better manage fatigue symptoms such as an increase of physical activity, and good sleep habits education.
Mental health meta-features: refers to aspects related to mental wellbeing such as symptoms of stress, anxiety, or low mood.

According to an embodiment of one or more paragraph(s) of this disclosure, the health recommender system may visualize objective and subjective data in a health-gram data where sensors' data providing objective data (e.g., sleep quality from a sensor, physical activity, heart rate) is annotated with subjective data (e.g., mood, perceived tiredness, perceived sleep quality) filled by the user using psychometrics. According to some embodiments, the different types of data are longitudinally integrated into a series of temporal vectors (e.g., hours, days, weeks, etc.), including semantic integration of related data. According to some embodiments, semantic integration may be facilitated by the use of semantic technologies such as ontologies and semantics networks.

According to an embodiment of one or more paragraph(s) of this disclosure, the health recommender system may include a user interface for presenting the health-gram and other data (e.g., recommendations, patient model, etc.).

One skilled in the art will realize the invention may be embodied in other specific forms without departing from the spirit or essential characteristics thereof. The foregoing embodiments are therefore to be considered in all respects illustrative rather than limiting of the invention described herein. In the foregoing detailed description, numerous specific details are set forth in order to provide an understanding of the invention. However, it will be understood by those skilled in the art that the invention can be practiced without these specific details. In other instances, well-known methods, procedures, and components, modules, units, and/or circuits have not been described in detail so as not to obscure the invention. Some features or elements described with respect to one embodiment can be combined with features or elements described with respect to other embodiments. The embodiments referred to above, and other embodiments, are described in detail in the next section.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention will be apparent from the following detailed description taken in conjunction with the accompanying drawings.

FIG. 1 is a schematic diagram showing an exemplary embodiment of a data space of the health recommender system including therapeutic content, user characteristics, and engagement data suitable for the implementation of an exemplary embodiment of the present disclosure.

FIG. 2 is a schematic diagram showing an exemplary embodiment of a process on how recommendations are generated and delivered to patients in accordance with an exemplary embodiment of the present disclosure.

FIG. 3 is a schematic diagram showing exemplary embodiments of a modeling process for the delivery of recommendations in the health recommender system suitable for the implementation of exemplary embodiments of the present disclosure.

FIG. 4 is a schematic diagram showing exemplary embodiments of a process on how recommendations are generated for a specific type of content in the health recommender system suitable for the implementation of exemplary embodiments of the present disclosure.

FIG. 5 is a schematic diagram showing an exemplary embodiment of ways in which the user feedback may be captured in the health recommender platform (i.e., how the system captures contextual engagement data) in accordance with an exemplary embodiment of the present invention.

FIG. 6 is a graph showing an exemplary embodiment of the visualization of objective and subjective health-gram data where sensors' data is annotated with subjective data in accordance with an exemplary embodiment of the present invention.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise.

It will be further understood that the terms “comprises” and/or “comprising” and/or “includes” and/or “including” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this disclosure belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing embodiments of the present disclosure, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion.

Nevertheless, the specification and claims should be read with the understanding that such combinations are entirely within the scope of the present disclosure.

In the following description, for purposes of explanation, numerous specific details are set forth in order to provide a thorough understanding of the present disclosure. It will be evident, however, to one skilled in the art that the present disclosure may be practiced without these specific details.

The present disclosure is to be considered as an exemplification of the invention and is not intended to limit the invention to the specific embodiments illustrated by the figures or description below.

Reference will now be made in detail to the present embodiments of the disclosure, certain examples of which are illustrated in the accompanying drawings.

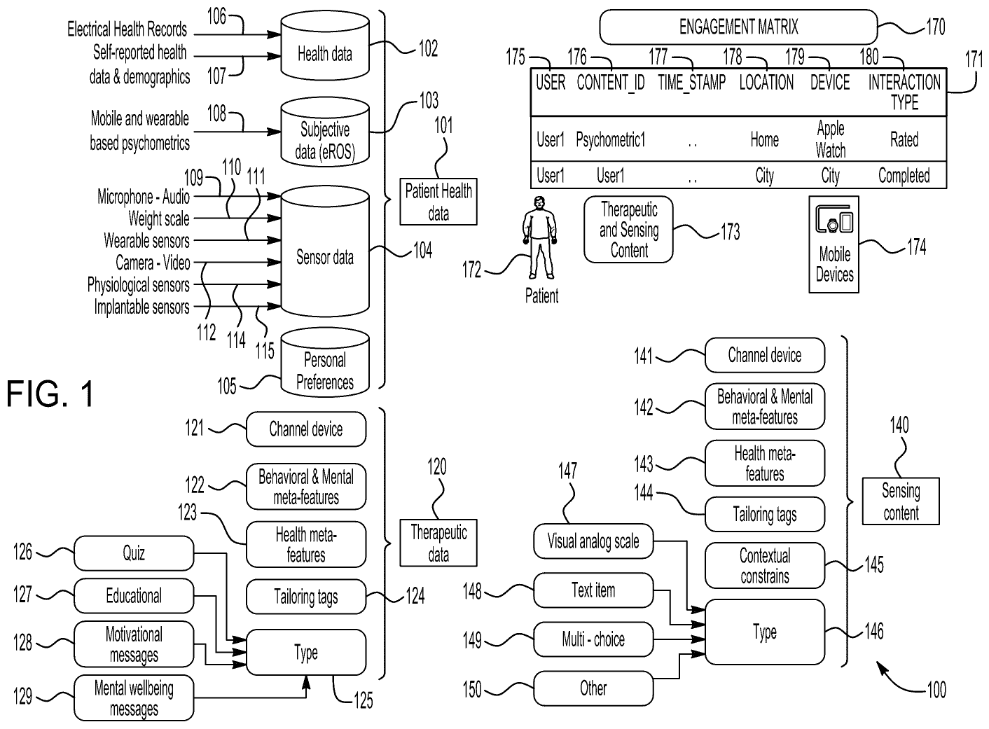

FIG. 1 is a schematic diagram illustrating an exemplary embodiment of a data space of a health recommender system 100 that may be used to implement certain aspects of the disclosed embodiments. The components and elements that are illustrated in FIG. 1 may vary consistent with the disclosed embodiments.

In one embodiment, the data space of the health recommender system 100 includes patient health data 101 which may include health data 102 aspects such as diagnosis, age, gender, treatments, and the like. The health data 102 can also include relevant demographic factors (e.g., age) and other social factors (e.g., educational level, social support, cultural preferences, family relations). For example, the health data 102 may include biopsychosocial models of data which consider the interconnection between biology, psychology, and socio-environmental factors and specifically how these aspects play a role in health. According to some embodiments, the health data 102 may be either self-reported 107 or coming from an electronic health record 106.

The patient health data 101 may further include subjective health data 103 which according to one embodiment, may include reported data 108 having been reported by the patient using mobile or wearable devices. For example, the patient may respond to a questionnaire by pressing a button on a wearable device, filling a questionnaire in a mobile device, or responding with a voice command to the wearable or mobile devices. The reported data 108 comes from the interaction of the user with sensing content 140, described in more detail below.

According to one embodiment, the subjective health data 103 may include electronic patient-reported outcomes (ePROs) which refer to a health outcome directly based on patient self-reporting.

According to some embodiments, the patient health data 101 may further include objective sensor data 104 coming from one or more sensors, which may include one or more microphones 109 for sensing audio signals, one or more weight scales 110, one or more wearable sensors 111 for acquiring data (e.g., sleep quality, physical activity, heart rate parameters, one or more cameras 112 for acquiring video and images (e.g., detected emotional expression from face images), one or more physiological sensors 114 (e.g., sensors for detecting galvanic response or sweating), or one or more implantable sensors 115 (e.g., sensors for continuous glucose monitoring).

According to some embodiments, the patient health data 101 may further include personal preferences 105 which may refer to preferences of the patient in terms of recommendations. In some embodiments, the personal preferences 105 may include aspects such as a desired tone of the content, health goals, timing, and frequency of the recommendations.

The data space of the health recommender system 100 as depicted in FIG. 1, may further include therapeutic data 120 which may be recommended to the patient. The therapeutic data 120 may include information on a channel device 121 used to deliver the content (e.g., wearable user interface, mobile user interface, etc.), behavioral and mental health meta-features 122 related to the health aspects that the content is addressing (e.g., emotional needs, reducing stress, etc.) or behavioral aspects (e.g., medication adherence, lifestyle recommendations).

According to some embodiments, the therapeutic data 120 may further include other health meta-features 123 addressing physical health aspects related to symptoms or health problems that the content has been designed for.

According to some embodiments, the therapeutic data 120 may further include tailoring tags 124 which may allow personalizing the content itself, such as adding goals to the therapeutic content, names, etc.

According to some embodiments, the therapeutic data 120 may also include a description of the type of therapeutic content 125 which may include, content such as education quizzes 126, educational content 127 such as audio/text/videos explaining self-management skills, motivations messages 128 and mental wellbeing exercises 129.

According to some embodiments, the data space of the health recommender system 100 may further include the sensing content 140 that may be used to describe subjective data from patients, such as psychometrics. According to some embodiments, the sensing content 140 may include metadata, for example channel device metadata 141 describing for which device the content is designed for, behavioral and mental meta-features 142 describing which related behavioral and mental health meta-features are being captured (e.g., mood level), health meta-features 143 describing information related to physical health meta-features (e.g., a question related to a particular diagnose) and, tailoring tags 144 such as “name” to provide the real name in the sensing content itself.

According to some embodiments, the sensing content 140 may further include any contextual constraints 145 (e.g., time of the data when the sensing content 140 is available, such as psychometrics to be filling right before going to sleep), and the type of sensing content 146 (e.g., type of psychometric, including its questions/items). The type of sensing content 146 may include visual analog scale information 147, text items 148, multiple-choice questions 149 and other relevant information 150.

According to some embodiments, the data space of the health recommender system 100 may further include an engagement matrix 170 comprising a table 171 of rows and columns wherein each row may include the logs of the interaction of the users 175 with the different types of content 176, that may include both sensing and therapeutic content 173 (i.e., the sensing and therapeutic content 173 is composed of therapeutic data 120 and sensing content 140) to engage a patient 172 via mobile devices 174. The table 171 of the engagement matrix 170 may further include a column describing the device type 179 on which the content has been accessed/interacted and its location 178. According to some embodiments, the table 171 of the engagement matrix 170 may further include a column with a TIME_STAMP field 177 and additionally the type of interaction 180 (e.g., rated, opened, completed).

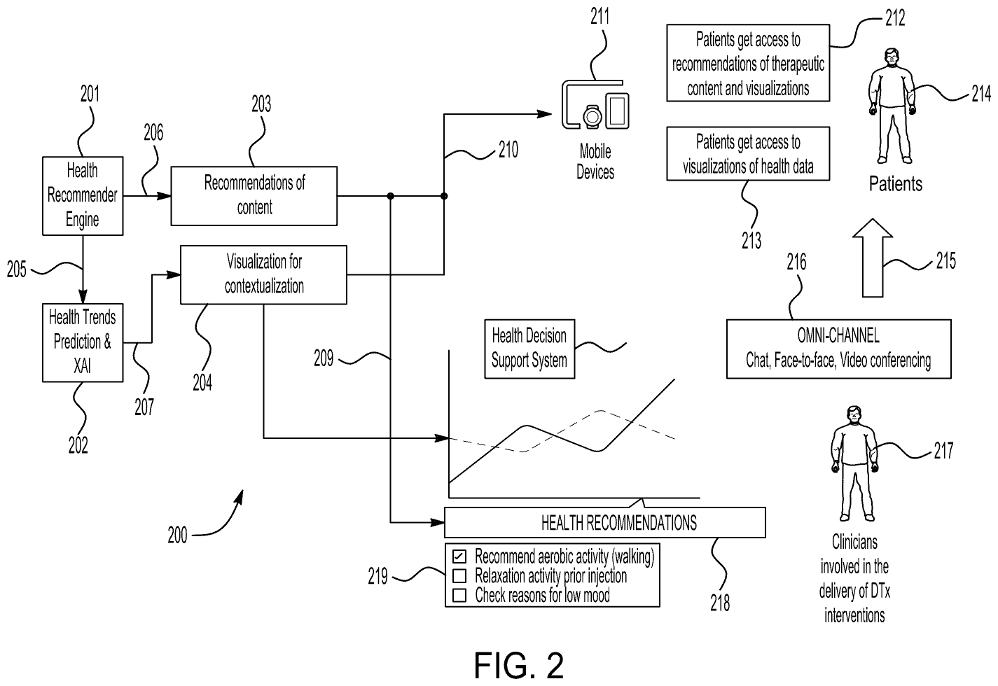

FIG. 2 is a diagram illustrating an exemplary embodiment of a process 200 describing the flow used to transmit recommendations to a patient 172, 214 in the health recommender system 100, that may be used to implement certain aspects of the disclosed embodiments.

According to some embodiments, the process 200 may be started with a step 206 of a health recommender engine 201 provides a recommendation 203 of either therapeutic or sensing content which is then transmitted in a step 210 to the patient 214 who has access to it via devices such as mobile phones 211 or wearable devices. According to some embodiments, the health recommender engine may be a computer implemented unit, comprising one or more processors which control the computer operation, memory and communication channels. According to some embodiments, one or more processors may be connected to cloud services. The processors can include different types of personal computers, servers, computing systems, communication devices, processors (e.g., digital signal processor (DSP), microcontrollers, field-programmable gate array (FPGA), application-specific integrated circuit (ASIC), etc.) and other electronic computing devices.

According to some embodiments, the recommendations 203 may be provided as a visualization of health data 213 for contextualization that may also be visible to the patient 214 in combination with the other recommendations 203 of therapeutic content. For example, and according to some embodiments, a recommendation 203 of content aiming at promoting physical activity as a way to reduce stress may include visualizations of the status of the weekly goal or visualizations of the prediction on the improvement of sleep quality if the users do increase physical activity.

In some embodiments, the recommendations 203 may also be sent in a step 209 to clinicians or health personnel 217 involved in the delivery of digital health interventions via a health decision support system 220. According to some embodiments, the health decision support system 220 combines the visualization of patient data and further allows the introduction of new data variables 219 about the patient 214, in addition to health recommendations 218 which the clinicians or health personnel 217 may convey to the patient 214 using multiple channels 216 and deliver in a step 215 to the patients 214. For example, and according to some embodiments, a clinician or health personnel 217 may get the suggestion to recommend the patient 214 mental wellbeing exercises prior to going to sleep to reduce stress and promote healthier sleep habits. The clinician or health personnel 217 then may give that recommendation 218 to the patient 214 using different communication channels 216 (e.g., phone, chat) and also include a link to content within the mobile application.

As may be apparent to the skilled in the art, the process 200 may use various types of communication channels 216 to deliver the health recommendations 218 to the patient 214. According to some embodiments, the communication channels 216 may include electronic communication channels (e.g., video conferencing, chats, emails, phone messages or phone calls, voice and/or video messages, etc.) or other channels as face-to-face meetings. In some embodiments, recommendations 218 may be delivered using a plurality of communication channels. For example, and according to some embodiments, a motivational message about healthy eating may be visualized differently in a small device (e.g., smartwatch) than in a bigger one (e.g., smartphone) and also adjust aspects such as text size or multimedia content.

According to some embodiments, the health recommender engine 201 may be connected to a health trends and prediction module 202, which may use artificial intelligence (e.g., Explainable Artificial Intelligence—XAI) to provide in a step 207 visualizations for the contextualization 204. In some embodiments, said visualizations for the contextualization 204 may be input in a step 208 to the health decision support system 220 which may provide graphical data to support the health recommendations.

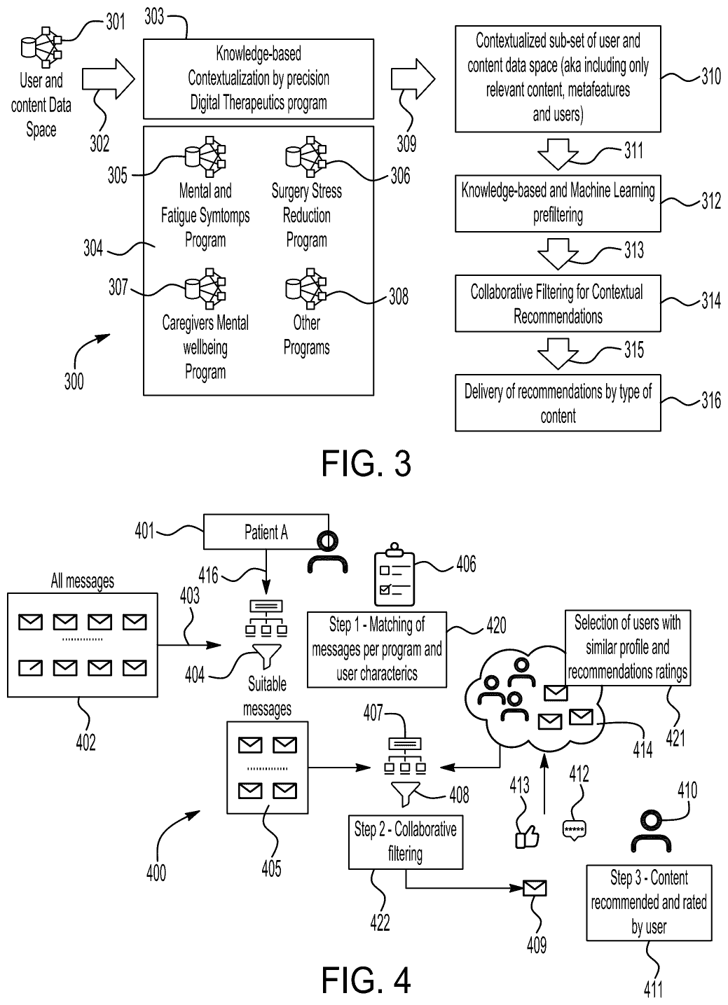

FIG. 3 is a diagram illustrating an exemplary embodiment of a modeling process 300 for the delivery of recommendations in the health recommender system 100, that may be used to implement certain aspects of the disclosed embodiments.

According to some embodiments, the modeling process 300 may start by using data stored in a common user and content data space 301. According to some embodiments, a step of pre-selection 302 of the related content/user data is done for each digital therapeutic program 303. This step of pre-selection 302 may apply multiple techniques (e.g., singular value decomposition (SVD) aiming at reducing the amount of information in the data-space 301 by selecting only content and/or data points relevant to the context of each therapeutics program 304. Following some examples of therapeutic programs 304:
a) Mental wellbeing and fatigue symptoms program 305: which may include therapeutic content aiming at helping patients in the fatigue symptoms management (e.g., Education for energy conservation and fatigue management) while promoting physical activity and using Cognitive-Behavioral Therapy content to improve sleep quality and improve mental wellbeing.
b) Surgery stress reduction program 306: This may focus on an eight weeks program enhancing lifestyle prior to the surgery (e.g., smoking cessation, physical activity) that is crucial to reduce future complications of surgery and promote recovery. This program may also include recommendations of content addressing mindfulness techniques to reduce surgery-related anxiety and post-surgery educational aspects.
c) Family Caregivers' mental wellbeing program 307: is a program in which its active period has 8 weeks where family caregivers can get recommendations on content to address the self-management of the chronic condition, in addition to content aiming at reducing the stress and mental wellbeing burden of the family caregivers.
d) Other programs 308: For example, Adhera Health Programs are designed to support specific needs across the patient journey of people living with chronic conditions. Users might transition from one program to another as their needs evolve. These transitions are largely based on the captured data about the health status of the user.

Relying on the meta-features of content and user profile 122, 123, 142, 143, a mapping of the relevant content to a given therapeutic program 304 is performed thus prefiltering content that is not relevant to the digital therapeutic program 303 in a step 309. According to some embodiments, this step 309 may result in a contextualized subset 310 which may be further pre-filtered in a step 311,312 using a combination of methods (e.g., rule-based, or machine learning) at the individual patient level. According to some embodiments, this may be further complemented with a collaborative filtering 313, 314 that identifies relevant recommendations by analyzing engagement (i.e., ratings) between similar patients and content.

According to some embodiments, the process 300 is completed when at least one recommendation is finally delivered in a step 315 to the patient 210, 215, across different types of devices depending on the type of the recommended content 316.

As may be apparent to the skilled in the art, these recommendations may be of different nature depending on their type. For example, some recommendations may have been configured to be provided only after a particular trigger (e.g., just reported low mood, after physical activity, etc.) or scheduled to be provided at particular times (e.g., recommendations of relaxation activities prior to taking an injection). The recommendations may be further personalized using tailoring tags 144 such as including names of clinicians or the patient, names of doctors, and weather.

Referring now to FIG. 4, a diagram illustrating an exemplary embodiment of a process 400 on how recommendations are generated for a specific type of content (e.g., motivational messages) in the health recommender system 100, that may be used to implement certain aspects of the disclosed embodiments.

According to some embodiments, the process 400 starts after a subset of relevant content, the contextualized subset 310 from the process 300, has been already selected for a given program following the steps illustrated in FIG. 3, using as example motivational messages 128, 402. According to some embodiments, these spaces of motivational messages 402 may be then filtered in a step 403 using a knowledge-based algorithm 404 that may be based on matching 420 between the motivational messages 402 and users' characteristics 406 (e.g., ontology-based rules, the semantic distance between models). That results in a small subset of suitable messages 405 for User A 401. Then, and according to some embodiments, the process 400 continues with the selection the suitable messages 405 using a method such as a collaborative filtering model 422 (e.g., item-based collaborative filtering, deep-learning-based models). According to some embodiments, the suitable messages 405 may be evaluated and ranked in steps 407 and 408 based on an analysis of previous ratings from similar users 421 (for example, similarity may be calculated using K-nearest neighbors' algorithm).

Once the previous step is completed, the selected messages 409 are then delivered to the user 410, this step may include some adaptations (e.g., adding name) using tailoring tags (124) as described in FIG. 1 above.

According to some embodiments, the user (e.g., patient) 410 may rate 412,413 at least one recommendation message in step 411. This activity may be done explicitly (i.e., giving explicit feedback, such as clicking a “like”) or using more actionable ratings (e.g., giving hearts to a recommendation, clicking “I will do it” icon, etc.). In some embodiments, the ratings can be also implicit (e.g., the message has been read). This rating may be then stored in the engagement matrix 170, where the rating may be a type of interaction 180, 171.

According to some embodiments, these ratings 412,413 are then stored by the health recommender system 100 and used in the future to identify similar patients 414. As may be apparent to the skilled in the art, the type of rating may vary depending on the type of content and program.

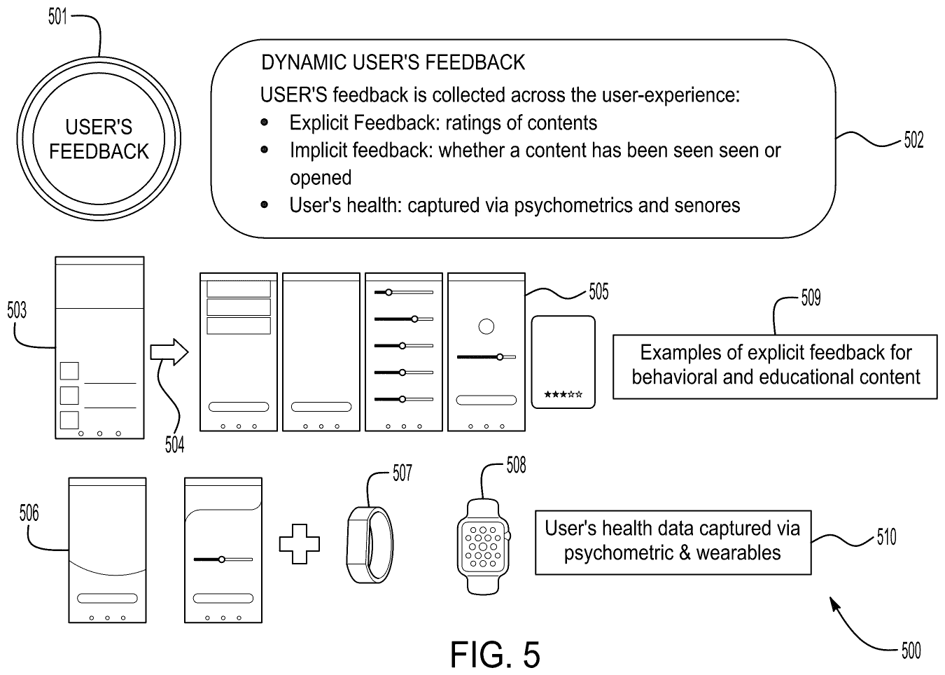

FIG. 5 is a diagram 500 illustrating exemplary ways in which the user feedback 501 may be captured in the health recommender system 100, that may be used to implement certain aspects of the disclosed embodiments.

For example, and according to some embodiments, therapeutic content 503 may include user's engagement feedback via quizzes, and/or questions about the content or ratings of the content. According to some embodiments of the present invention, the user's feedback 501 may include sensed data which may include mobile-based psychometrics 506, wearable data 507 psychometrics that can be captured directly by the user interface of the wearable device 508, and any other combination thereof. Different methods 502 for capturing user feedback 501 in the system which includes feedback related to behavioral and educational content 509, both explicit and implicit (which means no action needed by the user for providing feedback). An educational content 509 might include different ways 503 to elicit feedback in a step 504, such as rating an educational activity 505. Implicit feedback which requires no action by the user (e.g., opening a content without actually rating it), and also health data 510 of the user can be captured via sensors like wearables smartwatches 507 and 508 or mobile-based psychometrics 506.

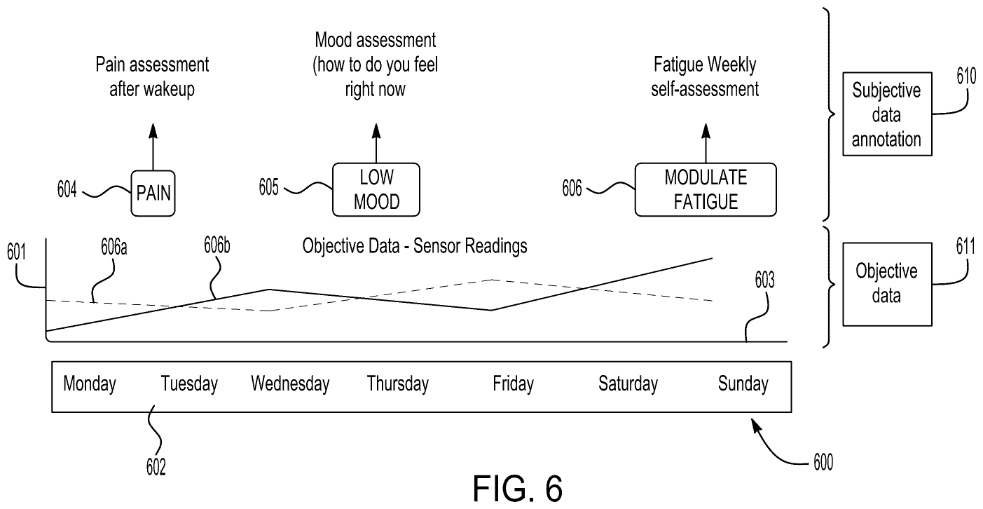

FIG. 6 illustrates an example of the visualization of an objective and subjective health-gram data 600 where sensors' data providing objective data is annotated with subjective data filled by the user using psychometrics. According to some embodiments, the different types of data are longitudinally integrated into a series of temporal vectors 606a, 606b, including semantic integration of related data (e.g. 604, 605, 606). According to some embodiments, semantic integration may be facilitated by the use of semantic technologies such as ontologies and semantics networks. For example, the x-axis 603 can represent the time (e.g., days of the week 602, hours of the day). In some embodiments, the continuous objective data 611 may be represented with lines (e.g., 606a, 606b) which may represent measurements such as movement or heart rate, in which the y-axis 601 will represent the values. In another example and according to some embodiments, in cancer-related fatigue, the visualization of the health data may include known contributing factors for fatigue that are objective (e.g., sleep quality from a sensor, physical activity, heart rate) with subjective data (e.g., mood, perceived tiredness, perceived sleep quality) that together with relevant clinical data can provide a more holistic visualization of the physical and mental health of the user. This integration may also need to consider the semantic similarity (e.g., relatedness between perceived sleep quality and objective sleep quality such as the number of sleep interruptions).

In some embodiments, subjective measurements 610 such as perceived pain 604, mood 605, perceived fatigue 606 may be represented as an overlay to the x-axis 603 depending on the measurement characteristics (e.g., how do you feel now, how did you feel the previous week).

The principles of the present disclosure, wherever applicable, are implemented as hardware, firmware, software, or any combination thereof. Moreover, the software is preferably implemented as an application program tangibly embodied on a program storage unit or computer-readable medium. The application program may be uploaded to and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such computer or processor is explicitly shown. In addition, various other peripheral units may be connected to the computer platform such as an additional data storage unit and a printing unit. The circuits described hereinabove may be implemented in a variety of manufacturing technologies well known in the industry including but not limited to integrated circuits (ICs) or SIP (system on chip) and discrete components that are mounted using surface mount technologies (SMT), and other technologies.

The system of the present disclosure may include, according to certain embodiments of the present disclosure, machine-readable memory containing or otherwise storing a program of instructions which, when executed by the machine, implements some or all of the system, methods, features, and functionalities of the present disclosure shown and described herein. Alternatively, or in addition, the system of the present disclosure may include, according to certain embodiments of the present disclosure, a program as above which may be written in any conventional programming language, and optionally a machine for executing the program such as but not limited to a general-purpose computer which may optionally be configured or activated in accordance with the teachings of the present disclosure. Any of the teachings incorporated herein may, wherever suitable, operate on signals representative of physical objects or substances.

The term “program” or “computer program” may include computer program code means for performing any of the methods shown and described herein when said program is run on at least one computer; and a computer program product, comprising a typically non-transitory-computer-usable or computer-readable medium, typically tangible, having a computer-readable program code embodied therein, said computer-readable program code adapted to be executed to implement any or all of the methods shown and described herein. Computer program code may be written in any suitable programming language and may be executed on a single computer system, or on a plurality of computer systems. The operations in accordance with the teachings herein may be performed by at least one computer specially constructed for the desired purposes or a general-purpose computer specially configured for the desired purpose by at least one computer program stored in a typically non-transitory computer-readable storage medium. The term “non-transitory” is used herein to exclude transitory, propagating signals or waves, but to otherwise include tangible items including any volatile or non-volatile computer memory technology suitable to the application.

Any suitable processor/s, display, and input means may be used to process, display e.g., on a computer screen or other computer output device, store, and accept information such as information used by or generated by any of the methods and systems that are shown and described herein: the above processor/s, display, and input means including computer programs, in accordance with some or all of the embodiments of the present disclosure. Any or all functionalities of the present disclosure shown and described herein, such as but not limited to operations within flowcharts, may be performed by anyone or more of: at least one conventional personal computer processor, workstation, or other programmable device or computer or electronic computing device or processor, either general-purpose or specifically constructed, used for processing; a computer display screen and/or printer and/or speaker for displaying; machine-readable memory such as optical disks, CDROMs, DVDs, Blu-ray, magnetic-optical discs or other discs; RAMs, ROMs, EPROMs, EEPROMs, Flash memories, magnetic or optical or other cards, for storing, and keyboard or mouse for accepting. Modules shown and described herein may include any one or combination or a plurality of a server, a data processor, a memory/computer storage, a communication interface, a computer program stored in memory/computer storage.

The term “process” as used in this application is intended to include any type of computation or manipulation or transformation of data (e.g., health data) represented as physical, e.g., electronic, phenomena which may occur or reside e.g., within registers and/or memories of at least one computer or processor. The term processor includes a single processing unit or a plurality of distributed or remote such units.

Any trademark occurring in the text or drawings is the property of its owner and occurs herein merely to explain or illustrate one example of how an embodiment of the present disclosure may be implemented.

Unless specifically stated otherwise, as apparent from the following discussions, it is appreciated that throughout the specification discussions, utilizing terms such as, “processing”, “computing”, “estimating”, “selecting”, “calculating”, “determining”, “generating”, “reassessing”, “classifying”, “generating”, “producing”, “registering”, “detecting”, “associating”, “obtaining” or the like, refer to the action and/or processes of at least one computer/s or computing system/s, or processor/s or similar electronic computing device/s, that manipulate and/or transform data represented as physical, such as electronic, quantities within the computing system's registers and/or memories, into other data similarly represented as physical quantities within the computing system's memories, registers or other such information storage, transmission or display devices. The term “computer” or “processor” should be broadly construed to cover any kind of electronic device with data processing capabilities, including, by way of non-limiting example, personal computers, servers, a computing system, communication devices, processors (e.g., digital signal processor (DSP), microcontrollers, field-programmable gate array (FPGA), application-specific integrated circuit (ASIC), etc.) and other electronic computing devices.

The present disclosure may be described, merely for clarity, in terms of terminology specific to particular programming languages, operating systems, browsers, system versions, individual products, and the like. It will be appreciated that this terminology is intended to convey general principles of operation clearly and briefly, by way of example, and is not intended to limit the scope of the invention to any particular programming language, operating system, browser, system version, or individual product.

Elements separately listed herein need not be distinct components and alternatively may be the same structure. A statement that an element or feature may exist is intended to include (a) embodiments in which the element or feature exists; (b) embodiments in which the element or feature does not exist; and (c) embodiments in which the element or feature exist selectively (e.g., a user may configure or select whether the element or feature does or does not exist). Any suitable input device, such as but not limited to a sensor, may be used to generate or otherwise provide information received by the apparatus and methods shown and described herein.

All examples and conditional language recited herein are intended for pedagogical purposes to aid the reader in understanding the principles of the present disclosure and the concepts contributed by the inventor to furthering the art and are to be construed as being without limitation to such specifically recited examples and conditions. Moreover, all statements herein reciting principles, aspects, and embodiments of the present disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

1. A method for supporting self-management of people with at least one chronic condition, and supporting the assessment of their psychical and mental conditions, wherein said method comprises a health recommender, the method carried out by at least one processor, said method comprising:
a) receiving, by the at least one processor, a plurality of first health parameters of a user, wherein said first health parameters denote the user's objective data, and wherein at least one of the first health parameters represents data measured by an external device;
b) receiving, by the at least one processor, a plurality of second health parameters of a user, wherein said second health parameters comprise at least one subjective measurement, and wherein said at least one subjective measurement denotes a perception of the user's subjective health;
c) receiving, by the at least one processor, additional data describing the interactions of the user with previous data generated by the health recommender;
d) receiving, by the at least one processor, content data, said content data comprising information relevant to the self-management of chronic conditions and characteristics of the recommendable content;
e) processing, by the at least one processor, the first and second health parameters to generate a first user model, wherein the first user model describes the user characteristics, and wherein the at least one processor has further access to additional user models previously generated for people with at least one chronic condition;
f) generating, by the at least one processor, at least one health recommendation to the user comprising content data, said recommendation based on the first user model, the content data and, the other user models;
g) selecting at least one recommendation for the user; and
h) providing the selected at least one recommendation to the user.

2. The method of claim 1 wherein said at least one processor is configured to communicate with at least one external device and wherein said external device is configured to exchange data with the at least one processor.

3. The method of claim 1 wherein said second health parameters comprise at least one subjective measurement, and wherein said subjective measurement comprises at least one of: psychometric parameters, questionnaire results, and the like.

4. The method of claim 1 wherein said processor has further access to previous recommendations provided by the health recommender and wherein said generating further comprises the interaction of the patient with at least one previous recommendation of the health recommender.

5. The method of claim 1 wherein said health recommender further comprises reducing the health data dimension, wherein said reducing comprises consideration of the unique individual health context of the user and health context of other users with similar characteristics, and wherein said reducing is performed before said generating.

6. The method of claim 1 wherein at least one function performed by the at least one processor further comprises the use of machine learning and artificial intelligence.

7. The method of claim 1, wherein said providing further comprises tailoring the selected recommendation to include personal characteristics of the user.

8. The method of claim 7 wherein said tailoring further comprises adapting the selected at least one recommendation to the user, by combining artificial intelligence models about symptoms predicted trends and their contributing factors.

9. The method of claim 1, wherein said first and second health parameters further comprise weighing factors and wherein said processing considers said weighing factors to generate the first user model.

10. A health recommender system to support the self-management of people with at least one chronic condition and to support the assessment of their psychical and mental conditions, the system comprising:
at least one processor comprising at least one communication channel configured to communicate with external devices to receive and store a plurality of first health parameters of a user, wherein said first health parameters denote the user's objective data and wherein at least one of the first health parameters represents data measured by an external device;
wherein said at least one processor is configured receive a plurality of second health parameters of a user, wherein said second health parameters comprise at least one subjective measurement;
wherein said at least one subjective measurement denotes a perception of the user's subjective health;
wherein said at least one processor is further configured to receive additional data describing the interactions of the user with previous data generated by the health recommender; and
wherein said at least one processor is further configured to receive content data comprising information relevant to the self-management of chronic conditions and characteristics of the recommendable content; and
a storage device configured to store and retrieve the plurality of first health parameters, the plurality of second health parameters of a patient, the content data, and the additional data;
wherein the at least one processor is configured to perform functions related to the first health parameters, the second health parameters, the content data and the additional data, said functions comprising:
a) receiving, by the at least one processor, a plurality of first health parameters of the user, wherein said first health parameters denote the user's objective data and wherein at least one of the first health parameters represents data measured by an external device;
b) receiving, by the at least one processor, a plurality of second health parameters of a user, wherein said second health parameters comprise at least one subjective measurement; wherein said at least one subjective measurement denotes a perception of the user's subjective health;
c) receiving, by the at least one processor, additional data describing the interactions of the user with previous data generated by the health recommender;
d) receiving, by the at least one processor content data, said content data comprising information relevant to the self-management of chronic conditions and characteristics of the recommendable content;
e) processing, by the at least one processor, the first and second health parameters to generate a first user model, wherein the first user model describes the user characteristics; and wherein the at least one processor has further access to additional user models previously generated for people with at least one chronic condition;
f) generating, by the at least one processor, at least one health recommendation to the user comprising content data, said recommendation based on the first user model, the content data and, the other user models;
g) selecting at least one recommendation for the user; and
h) providing the selected at least one recommendation to the user.

11. The system of claim 10 wherein said at least one processor is configured to communicate with at least one external device and wherein said external device is configured to exchange data with the at least one processor.

12. The system of claim 10 wherein said second health parameters comprise at least one subjective measurement, and wherein said subjective measurement comprises at least one of: psychometric parameters, questionnaire results, and the like.

13. The system of claim 10 wherein said processor has further access to previous recommendations provided by the health recommender and wherein said generating further comprises the interaction of the patient with at least one previous recommendation of the health recommender.

14. The system of claim 10 wherein said health recommender further comprises reducing the health data dimension, wherein said reducing comprises consideration of the unique individual health context of the user and health context of other users with similar characteristics, and wherein said reducing is performed before said generating.

15. The system of claim 10 wherein at least one function performed by the at least one processor further comprises the use of machine learning and artificial intelligence.

16. The system of claim 10, wherein said providing further comprises tailoring the selected recommendation to include personal characteristics of the user.

17. The system of claim 16 wherein said tailoring further comprises adapting the selected at least one recommendation to the user, by combining artificial intelligence models about symptoms predicted trends and their contributing factors.

18. The system of claim 10, wherein said first and second health parameters further comprise weighing factors and wherein said processing considers said weighing factors to generate the first user model.

19. A non-transitory computer readable storage medium in a system comprising at least one processor with at least one communication channel configured to communicate with external devices, a memory and, access to a storage device to store and retrieve data, wherein said computer readable storage medium stores at least one readable program, wherein said at least one program supports the self-management of people with at least one chronic condition and supports the assessment of their psychical and mental conditions, and when said at least one program is executed by the at least one processor causes the at least one processor to:
a) receive, by the at least one processor, a plurality of first health parameters of a user, wherein said first health parameters denote the user's objective data and wherein at least one of the first health parameters represents data measured by an external device;
b) receive, by the at least one processor, a plurality of second health parameters of the user, wherein said second health parameters comprise at least one subjective measurement; wherein said at least one subjective measurement denotes a perception of the user's subjective health;
c) receive, by the at least one processor, additional data describing the interactions of the user with previous data generated by the health recommender;
d) receive, by the at least one processor, content data, said content data comprising information relevant to the self-management of chronic conditions and characteristics of the recommendable content;
e) process, by the at least one processor, the first and second health parameters to generate a first user model, wherein the first user model describes the user characteristics; and wherein the at least one processor has further access to additional user models previously generated for people with at least one chronic condition;
f) generate, by the at least one processor, at least one health recommendation to the user comprising content data, said recommendation based on the first user model, the content data and, the other user models;
g) select at least one recommendation for the user; and
h) provide the selected at least one recommendation to the user.
